(19) United States
(12) Patent Application Publication
(10) Pub. No.: US 2015/0133721 A1
(43) Pub. Date: May 14, 2015

(54) HEART ASSIST APPARATUS AND METHOD OF USE THEREOF

(71) Applicant: Michael D. Roth, Scottsdale, AZ (US)

(72) Inventor: Michael D. Roth, Scottsdale, AZ (US)

(21) Appl. No.: 14/078,254

(22) Filed: Nov. 12, 2013

(51) Int. Cl. A61M 1/12; A61M 1/10

(52) U.S. Cl. CPC A61M 1/122; A61M 1/1086
USPC 600/17; 600/16

(57) ABSTRACT

The invention comprises a heart assist device. In one embodiment, the heart assist device comprises a compressible sleeve about a body part, where one or more portions of the sleeve compress to provide a force on blood generating a flow or pulse of blood in the human circulatory system. In one case, the sleeve is external to the body, such as circumferentially about a limb or about a body extremity. In another case, the sleeve is internal to the body, such as circumferentially about an artery or vein. In still another case, two or more sleeves cooperatively function in parallel and/or in series. The heart assist device is optionally configured to auto-start in an emergency system, is in on-demand or in continuous contact with an emergency response system, and/or is linked to a medical professional's system.

### CROSS-REFERENCE TO RELATED APPLICATION

This application claims the benefit of U.S. provisional patent application No. 61/727,586, filed Nov. 16, 2012, all of which is incorporated herein in its entirety by this reference thereto.

### TECHNICAL FIELD OF THE INVENTION

The present invention relates to a heart assist apparatus and method of use thereof.

### DESCRIPTION OF THE RELATED ART

Heart transplants are traumatic, expensive, and have a history of short term and especially long term failure.

Although the heart is conceptually a pump, the heart is a complex organ embodying chemical, physical, and mechanical subtleties. Indeed, a synthetic heart replacement, which would replace heart transplants, continues to be an unreached goal of modern medicine. Multiple issues remain, including power, size, durability, replacement need, damage to circulating blood, the need for a mimicked pulse, and the natural process of the body's foreign body rejection.

In lieu of a heart replacement, ventricular assist devices (VAD) are envisioned, which do not replace the human heart but do alleviate strain on the heart. A ventricular assist device is used to take up much of the function of a heart, which reduces stress on the heart and ultimately lowers the need for heart transplants.

### Problem Statement

What is needed is an affordable, readily usable, and effective heart assist device and/or an artificial heart.

### SUMMARY OF THE INVENTION

The invention comprises a heart assist apparatus and method of use thereof.

### BRIEF DESCRIPTION OF THE DRAWINGS

A more complete understanding of the present invention is derived by referring to the detailed description and claims when considered in connection with the Figures, wherein like reference numbers refer to similar items throughout the Figures.

FIG. 1 illustrates major components and operation of a heart assist device;

FIG. 2A and FIG. 2B illustrate an electroactive polymer and an electroactive polymer sleeve, respectively;

FIGS. 3(A-D) illustrate a series of electroactive sleeve segments operating in time series on a body part at a first time, FIG. 3A; at a second time, FIG. 3B, at a third time, FIG. 3C; and at a fourth time, FIG. 3D; and

FIG. 4 illustrates two or more cooperatively operating heart assist devices.

Elements and steps in the figures are illustrated for simplicity and clarity and have not necessarily been rendered according to any particular sequence. For example, steps that are performed concurrently or in different order are illustrated in the figures to help improve understanding of embodiments of the present invention.

### DETAILED DESCRIPTION OF THE PREFERRED EMBODIMENTS

The invention comprises a heart assist device.

In one embodiment, the heart assist device comprises a compressible sleeve about a body part, where one or more portions of the sleeve compress to provide a force on blood generating a flow or pulse of blood in the human circulatory system. In one case, the sleeve is external to the body, such as about a limb or a body extremity. In another case, the sleeve is internal to the body, such as about an artery or vein. In still another case, two or more sleeves cooperatively function in parallel and/or in series.

### Heart Assist Device

A blood circulation device 100 is optionally an artificial heart or a heart assist device. For clarity of presentation and without loss of generality, the heart assist device is further described herein. However, the elements of the heart assist device 110 and their uses also apply to use as an artificial heart.

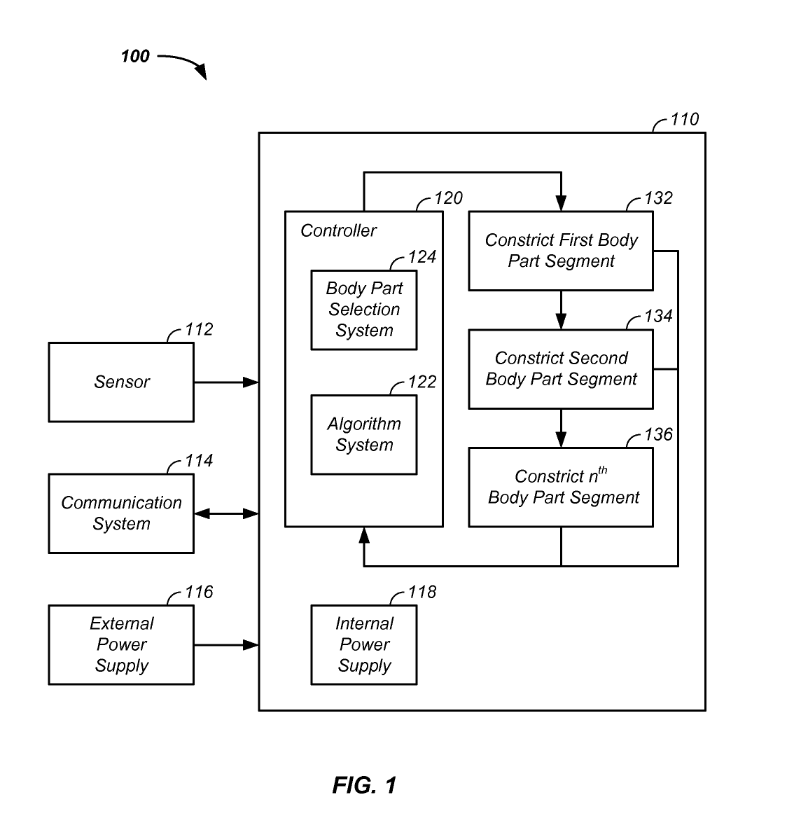

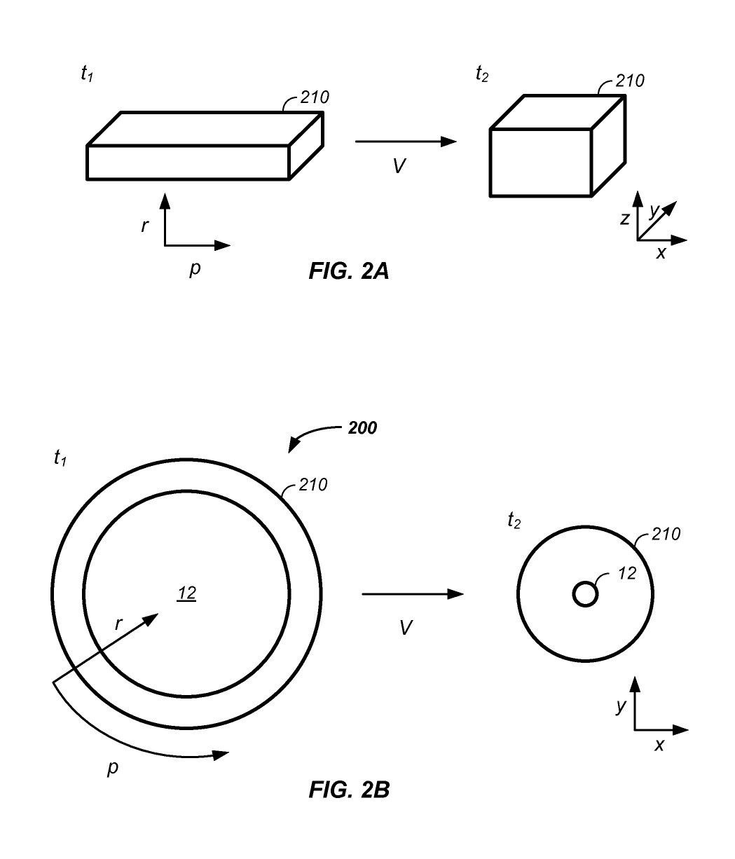

Referring now to FIG. 1 and FIG. 2B, an example of a heart assist device 110 is provided. Generally, the heart assist device 110 includes a controller 120 and a sleeve 200, where an inner cross-sectional area 134 of the sleeve 200 is controlled by the controller 120 as a function of time. As the inner diameter of the sleeve 200 about the body part constricts, blood is forced from the body part to an adjoining body part, such as through the circulatory system.

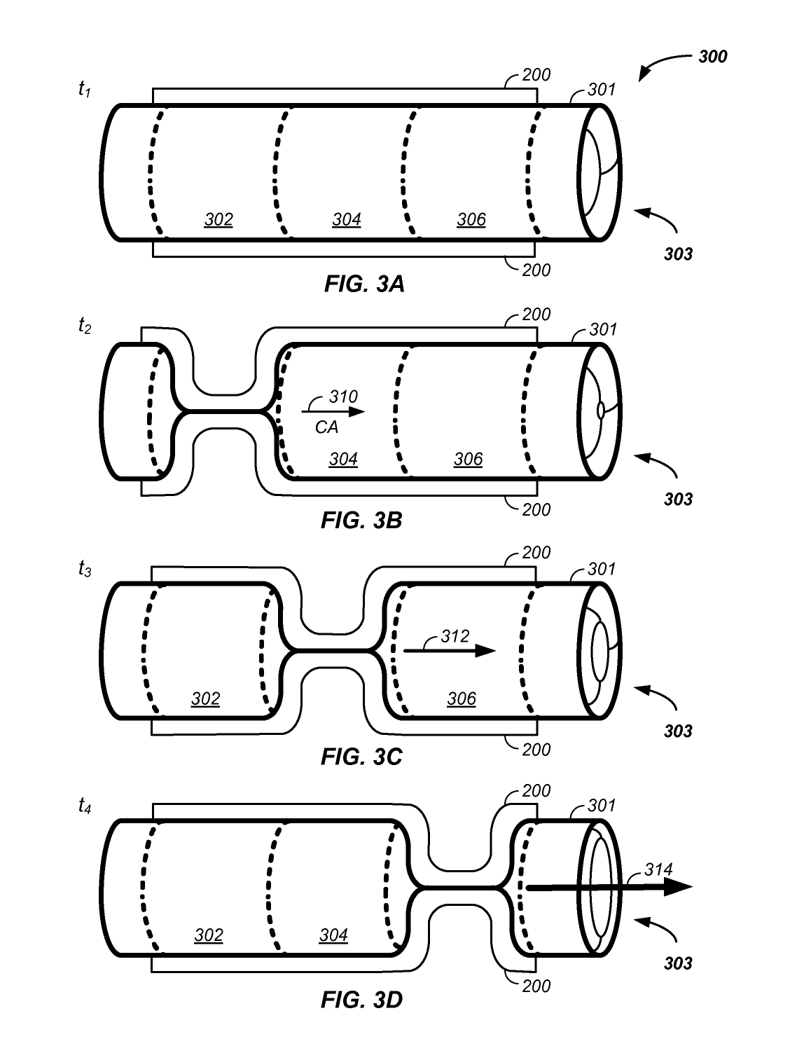

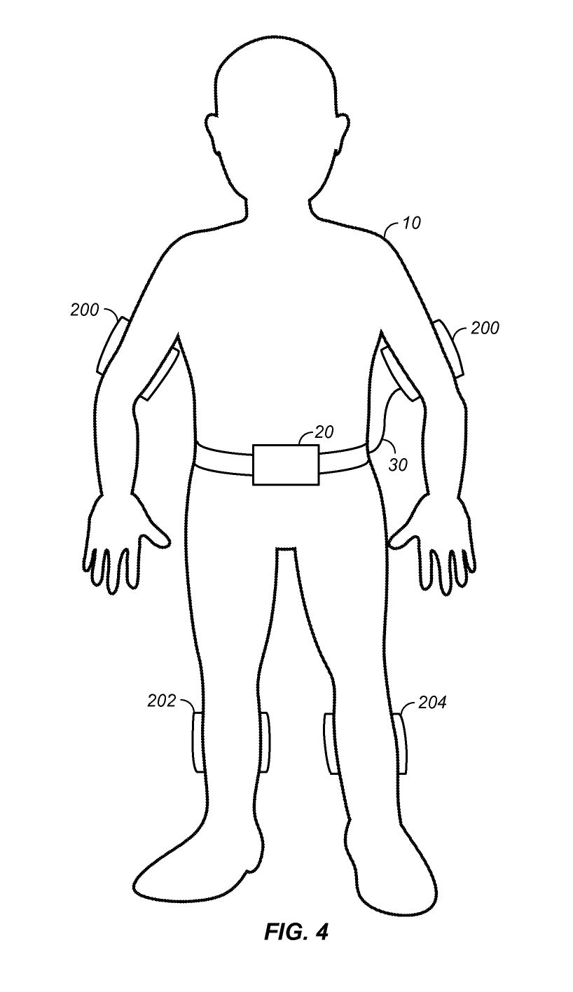

Still referring to FIG. 1, the controller 120 of the heart assist device 110 controls: (1) timing of constriction of the sleeve 200 and (2) position of constriction of the sleeve 200. In a first case, the controller 120 controls constriction of the sleeve 200 about a body part. In a second case, the controller 120 controls a series of constrictions of a series of segments of the sleeve 120 about a series of segments about a single body part to force blood in a forward direction through the circulatory system of the body. For example, the sleeve segments are serially constricted about an artery along the axis of blood flow to force blood in the artery through the circulatory system of the body. The second case is further described, infra, when referring to FIG. 3. In a third case, the controller 120 controls a set of constrictions of a set of sleeves about two or more body parts. For example, two or more sleeves 200 are positioned on the arms and/or legs of the body and the controller constricts the individual sleeves at a single time, in a rhythm, in a repeating and/or repetitive pattern, in series, in parallel and/or under the control of the controller 120. The third case is further described, infra, when referring to FIG. 4. In a fourth case, the heart assist device 110 constricts a series of segments of each of two or more sleeves 200, which is a combination of the second case with the third case. Generally, the controller directs constriction of one or more sleeves about a first body segment 132, about a second body segment 134, and/or about an n^{th} body segment 136 as a function of time, where n is a positive integer. Optionally, two or more sleeve segments are constricted at essentially the same time, such as within less than 1, 0.5, 0.25, or 0.1 seconds apart; having overlapping constriction periods; and/or have time periods of initiation of constriction at least 0.05, 0.1, 0.25, or 0.5 seconds apart.

Generally, in addition to the controller 120 with an optional algorithm system 122 and/or a body part selection system 124, the heart assist device 110 optionally includes any of: a sensor 112, such as a hemodynamic sensor and/or an electrodynamic sensor, a communication system 114, an external power supply 116, and/or an internal power supply 118. The sensor 112 and the communication system 114 are preferably external to the body, but are optionally implanted into the body.

Referring now to FIG. 2, the sleeve 200 of the heart assist device 110 is further described. Herein for clarity of presentation and without loss of generality, an electroactive polymer is used to illustrate function of the sleeve. For example, the sleeve 200 is optionally a mechanical peristaltic pump, a pneumatic system, and/or an artificial muscle. The electroactive polymer is optionally an ionic polymer-metal composite, a dielectric polymer, an ionic polymer, an electrostrictive polymer, and/or a liquid crystalline polymer. Optionally, the electroactive polymer is configured as fibers or fiber bundles formed into a ring to enhance range of motion. Generally, the sleeve 200 uses any material, mechanical system, biomechanical system, and/or electrodynamic system that changes shape as a function of time to move blood in the circulatory system of the body. Similarly, the sleeve 200 material is optionally any natural material, synthetic material, and/or polymer that changes shape as a function of time to move blood in the circulatory system of the body.

Referring now to FIG. 2A, an electroactive polymer 210 is further described. An electroactive polymer changes shape when stimulated by an electric field.

Typically, an electroactive polymer is capable of a large amount of deformation while sustaining large forces. In FIG. 2A, the electroactive polymer 210 is depicted in a first geometry at a first point in time, t₁, and in a second geometry at a second point in time, t₂, after application of a voltage, V, to the electroactive polymer 210. In the geometry depicted, the electroactive polymer reduces length along the x-axis and increases thickness along the z-axis after application of the electric field. For clarity, the x-axis is also labeled as the perimeter, p, axis and the z-axis is also labeled as the radius, r, axis. While the electroactive polymer 210 is depicted as increasing in thickness and shrinking in length as a function of an applied electric field, generally independent control of each axis shape of the electroactive polymer 210 is made possible through the chemical choice of monomer units of the polymer, control of the electric field, and manufacturing process. In addition, while the applied force to the electroactive polymer 210 is depicted as a voltage, optionally the applied force is any current, pressure, magnetic field, or induced field acting on the sleeve material that results in a changed shape as a function of time.

Referring now to FIG. 2B, the electroactive polymer depicted in FIG. 2A is formed as a ring or as the sleeve 200 about a body part 12. As orientated, the x-axis of FIG. 2A is now the perimeter axis, p, in FIG. 2B and the z-axis of FIG. 2A is now the radius axis in FIG. 2B. With application of the electric field to the electroactive polymer 210, the x-axis reduces in size. The reduced x-axis length shrinks the outer perimeter of the sleeve 200. Similarly, the z-axis of FIG. 2A is now the radius axis, r, of the sleeve 200 in FIG. 2B. With application of the electric field to the electroactive polymer 210, the z-axis increases in size. The increased z-axis length shrinks the inner perimeter of the sleeve 200. The decreased perimeter of the sleeve 200 and/or the increased radial thickness of the sleeve 200 reduces or constricts the size of the body part 12 within the sleeve. For example, an artery is constricted by the electroactive polymer upon application of an electric field to the electroactive polymer 210. With removal of the electric field, the electroactive polymer will revert back to its original shape. The process of cyclical, periodic, rhythmic, or controlled time series application of the electric field to the electroactive polymer 210 by the controller 120 is repeated in a manner resulting in the circulation of blood in the circulatory system of the body.

Referring now to FIG. 3, a vein 300 having an outer wall 301, a circulation axis 310, CA, and a valve 303 is illustrated, where the vein 300 is also representative of an artery or an extremity of the body, such as an arm or leg. The sleeve 200 or electroactive polymer 210 in the shape of a sleeve is circumferentially positioned about the artery 300, such as in proximate contact with the wall 301 of the artery 300. Referring now to FIG. 3A, at a first point in time, t₁, the sleeve 200 is maintained in a non-constricted state, which is optionally a relaxed state of the electroactive polymer 210. Referring now to FIG. 3B, at a second point in time, t₂, a first segment 302 of the sleeve 200 is constricted, which forces blood forward along the circulation axis at a first velocity or pressure 310. Optionally, at a third point in time, t₃, a second segment 304 of the sleeve 200 is constricted, which, in combination with the prior constriction of the first segment 302, forces the blood forward along the circulation axis at a second velocity or pressure 312. Optionally, at a fourth point in time, t₄, a third segment 306 of the sleeve 200 is constricted, which, in combination with the prior constriction of the first segment 302 and second segment 304, forces the blood forward along the circulation axis at a third velocity or pressure 314. Optionally, the sleeve 200 is positioned prior to the valve 303, such as within less than 1, 2, 3, 4, or 5 inches of the valve 303 to use the valve 303 as a natural backflow prohibited to the blood flow. Two of more segments are optionally simultaneously constricted. For example, the first segment 302 is optionally constricted until the second segment 304 is constricted to force the displaced blood forward along the circulation axis. Generally, the sleeve 200 optionally contains n segments, where n is a positive integer of at least one and the sleeve is sequentially constricted along the circulation axis 310 through the n segments.

Referring now to FIG. 4, the heart assist device 110 is depicted as using two or more sleeves 200 on arms and legs of a subject 10. However, the description of use of two or more sleeves, as illustrated about arms and legs of the subject 10, also applies to the use of two or more sleeves about arteries or veins of the subject 10.

Still referring to FIG. 4, the heart assist device 110 need not replace a heart of the subject 10. The heart assist device 110 optionally assists the heart for a short duration, such as for minutes or hours as in a medical emergency when the heart stops, or longer term such as days, weeks, months, or years to aid a failing heart. As such, the sleeve 200 is optionally worn externally to the subject 10, such as along an arm or leg of the subject 10. For clarity of presentation, an example of multiple sleeves on legs is used. A first sleeve 202 compresses a portion of a first leg and/or foot which forces the pooling or poorly circulating blood up the first leg and/or foot in the circulatory system. The resulting force on the blood may be sufficient to be used independently. Optionally, a second sleeve 204 about a second leg or foot is also similarly used. The controller 120 optionally constricts the first sleeve 202 and the second sleeve 204 at the same time, within one, two, or three seconds of each other, or at different times. Similarly, the first sleeve 202 and the second sleeve 204 are optionally worn on the same leg, a leg and an arm, or on two arms. Any number of sleeves 200 are optionally used and the controller optionally constricts any one or any combination of the sleeves at the same or different times to achieve circulating blood flow in the subject 10. An optional embodiment of the sleeve 200 is in the form of a sock. Optionally, the sensor 112 is integrated into the sock.

Still referring to FIG. 4, the blood circulation device optionally uses a power supply and/or user communication system worn as a belt 20 that is optionally directly wired using a wire 30 to the sleeve 200.

As described, supra, the blood circulation device 100 is optionally used with a sensor 112. The electrodynamic and/or hemodynamic sensor is optionally used to provide information about pulse, temperature, and/or blood pressure to the controller 120 where the algorithm system 122 determines a need to increase, maintain, or decrease the blood flow.

Optionally, a sensor is used as part of the heart assist device, such as to determine timing of a function related to the heart, such as timing of a blood pulse, measurement of a ventricular stroke volume, measurement of a ventricle filling rate, determination of a radial pulse, and/or determination of a radial blood flow. Optionally, the sensor or set of sensors is used to time function of the ventricular assist device, such as to time initiation of an induced pulse, median time of an induced pulse, or mean time of an induced pulse to lag a pulse initiation of the heart by more than 0.01, 0.05, 0.1, 0.2, 0.3, 0.5, 0.75, or 1 second to time the assist of the induced pulse with the heart pulse passing through the ventricular assist device pumping mechanism, so as to enhance and not impede the heart pulse.

### Emergency Use

The sensor 112 or set of sensors, such as the electrodynamic and/or hemodynamic sensor are also optionally configured with the controller for use in an emergency situation, such as with an arrhythmia or with stoppage of the heart. In such an event, the sensor 112 is used to detect the emergency situation and to initiate start-up of the blood circulation device 100. In this case, the blood circulation device 100 was worn by the subject 10 in the event of an emergency. For example, the blood circulation device 100 is optionally developed into socks and worn daily in old age in the event of a heart attack. In another example, the blood circulation device 100 is worn by a patient as a security measure during an operation or while sleeping. In yet another example, the blood circulation device is configured with an audible alarm and/or a verbal alarm notifying the user and/or people proximate the user of a prognosticated or current emergency medical situation. For example, the sensor 112 or set of sensors optionally determines a partial circulatory system blockage, abnormal oxygen levels in the blood, and/or a blood pressure rise while at rest and prognosticates a heart event due to decreased oxygen to the heart muscles.

### Communication System

Generally, a communication system operating in conjunction with the heart assist device 110 communicates state of the subject 10 to the subject 10 and/or to a remote system, such as to an emergency network system and/or to a medical practitioner.

In one case, the communication system is a link to a smartphone. The smartphone herein also refers to a feature phone, a tablet, a phablet, a mobile phone, a portable phone, and/or a cell phone. The smartphone contains a number of hardware and software features, which are optionally usable in combination with the blood circulation device 100, such as a hardware port, a communication system, a user interface system, a global positioning system, a memory system, a secure section, an identification system, and/or a power inlet or power supply.

The hardware port of the smartphone typically optionally contains one or more electro-mechanical connectors designed to physically link to the blood circulation device. Examples of connectors include a power supply port, a universal serial bus (USB) port, an audio port, a video port, a data port, a port for a memory card, and a multi-pin connector, such as a 30-pin connector. Further, integration of the heart assist device 110 with a smartphone reduces need for an integrated computer system and communication system. Still further, integration of the heart assist device 110 with a smartphone provides a back-up or redundant system, which is helpful in a life-saving/life-maintaining apparatus.

Each of the communication system, the personal communication device, the user interface system, the global positioning system, and/or the memory of the smartphone is optionally used as part of the blood circulation device 100. In a first example, the subject 10 uses the smartphone to call an authority system to report the individual's location, using the communication system, user interface system, and global positioning system, where the smartphone is used to confirm identity, medical state, and position of the individual. In a second example, the cell phone automatically communicates position and medical state of the individual to an emergency system without interaction of the individual 10. Herein, for clarity of presentation the smartphone is used to describe a generic digital communication device, such as a phone, a tablet computer, and/or a computer.

### Personal Monitor

In another embodiment, the blood circulation device 100, described supra, is used as a part of a process of relaying personal data to an external network. For example, a sensor is used to read a body parameter of the subject 10 and to relay the data directly and/or through the communication device to an external network. For example, the blood circulation device and smartphone combination is used as part of a personal health monitoring system. In the personal health monitoring system, the user 10 wears the blood circulation device 100 and data from the sensor 112 and/or the blood circulation device 100 is sent through the communication device to a remote service, such as a health monitoring company, the user's personal computing system, a medical monitoring service, friends, family, and/or an emergency response agency. Examples of the sensor 112 include any of: an alcohol monitor, a drug monitor, a temperature monitor, a pacemaker monitor, a heart rate monitor, a blood pressure monitor, an electrode affixed to a body part, a force meter, a temperature probe, a pH reader, a hydration monitor, or a biomedical sensor element. For example, the wearable biomedical sensor monitors a pacemaker and in the event of an abnormality relays the abnormality and location of the individual through the communication device to a remote service, such as to a dispatcher, for medical service and/or to a medical professional.

Still yet another embodiment includes any combination and/or permutation of any of the elements of any of the embodiments described herein.

The particular implementations shown and described are illustrative of the invention and its best mode and are not intended to otherwise limit the scope of the present invention in any way. Indeed, for the sake of brevity, conventional manufacturing, connection, preparation, and other functional aspects of the system may not be described in detail. Furthermore, the connecting lines shown in the various figures are intended to represent exemplary functional relationships and/or physical couplings between the various elements. Many alternative or additional functional relationships or physical connections may be present in a practical system.

In the foregoing description, the invention has been described with reference to specific exemplary embodiments; however, it will be appreciated that various modifications and changes may be made without departing from the scope of the present invention as set forth herein. The description and figures are to be regarded in an illustrative manner, rather than a restrictive one and all such modifications are intended to be included within the scope of the present invention. Accordingly, the scope of the invention should be determined by the generic embodiments described herein and their legal equivalents rather than by merely the specific examples described above. For example, the steps recited in any method or process embodiment may be executed in any order and are not limited to the explicit order presented in the specific examples. Additionally, the components and/or elements recited in any apparatus embodiment may be assembled or otherwise operationally configured in a variety of permutations to produce substantially the same result as the present invention and are accordingly not limited to the specific configuration recited in the specific examples.

Benefits, other advantages and solutions to problems have been described above with regard to particular embodiments; however, any benefit, advantage, solution to problems or any element that may cause any particular benefit, advantage or solution to occur or to become more pronounced are not to be construed as critical, required or essential features or components.

As used herein, the terms “comprises”, “comprising”, or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present invention, in addition to those not specifically recited, may be varied or otherwise particularly adapted to specific environments, manufacturing specifications, design parameters or other operating requirements without departing from the general principles of the same.

Although the invention has been described herein with reference to certain preferred embodiments, one skilled in the art will readily appreciate that other applications may be substituted for those set forth herein without departing from the spirit and scope of the present invention.

1. A method for aiding function of a heart of a patient, comprising the steps of:
providing a ventricular assist device, comprising:
a controller;
a first electroactive polymer sleeve segment circumferentially positioned about a portion of a first body part of the patient; and
a second electroactive polymer sleeve segment circumferentially positioned about a portion of a second body part of the patient; and
using said controller, timing constriction of both: (1) said first electroactive polymer sleeve segment and (2) said second electroactive polymer sleeve segment to aid the heart in circulation of blood in the patient.

2. The method of claim 1, further comprising the steps of:
using a heart sensor to determine a signal related to a heart-beat of the patient, said heart sensor comprising at least one of: an electrodynamic sensor and a hemodynamic sensor; and
using the signal in said step of timing constriction.

3. The method of claim 2, further comprising the steps of:
positioning said first electroactive polymer sleeve segment about a portion of a leg of the patient; and
positioning said second electroactive polymer sleeve segment about the portion of the leg of the patient within three inches of said first electroactive polymer sleeve segment, said second electroactive polymer sleeve segment within three inches of a vein valve in the patient,
wherein said step of timing constriction first constricts said first electroactive sleeve segment and within one-half second subsequently constricts said second electroactive sleeve segment.

4. The method of claim 3, wherein said step of positioning said first electroactive polymer further comprises the step of:
implanting the first electroactive polymer sleeve about a vein of the patient.

5. The method of claim 2, further comprising the steps of:
positioning said first electroactive polymer sleeve segment externally about a first leg of the patient; and
positioning said second electroactive polymer sleeve segment externally about a second leg of the patient, said second electroactive polymer sleeve segment within three longitudinal inches of a vein valve in the patient,
wherein said step of timing essentially simultaneously constricts said first electroactive polymer sleeve segment and said second electroactive polymer sleeve segment.

6. The method of claim 1, wherein said step of timing constriction, further comprises the step of:
serially timing relaxation of said second electroactive polymer, constriction of said first electroactive polymer, and constriction of said second electroactive polymer.

7. The method of claim 2, further comprising the step of:
lagging said step of timing constriction by one-tenth of a second to one second from determination of the heart-beat.

8. The method of claim 2, further comprising the step of:
using said signal, initiating start-up of blood circulation using said ventricular assist device upon determination of an emergency stoppage of the heart.

9. The method of claim 8, further comprising the step of:
upon detection of the emergency stoppage of the heart, using a personal communication device of the patient to notify at least one of:
an emergency response team; and
a relative.

10. The method of claim 9, further comprising the step of:
communicating geographic position of the patient and state of the patient to the emergency response team.

11. The method of claim 1, further comprising the step of:
repetitively constricting an inner cross-section aperture circumferentially surrounded by said first electroactive polymer segment.

12. The method of claim 1, further comprising the step of:
using said controller to control position of constriction of said first electroactive polymer sleeve segment.

13. The method of claim 1, further comprising the step of:
repetitively reducing an inner perimeter of an annular form of said electroactive polymer sleeve segment using a polymer deforming electric field.

14. The method of claim 2, further comprising the steps of:
using said sensor to prognosticate heart failure up to three hours before onset of a heart attack; and
communicating the prognosticated heart failure to a remote medical response team using a personal communication device of the patient.

15. An apparatus for aiding function of a heart of a patient, comprising:
a ventricular assist device, comprising:
a controller;
a first electroactive polymer sleeve segment circumferentially positioned about a portion of a first body part of the patient; and
a second electroactive polymer sleeve segment circumferentially positioned about a section of a second body part of the patient,
wherein said controller, times constriction of both: (1) said first electroactive polymer sleeve segment and (2) said second electroactive polymer sleeve segment to aid the heart in circulation of blood in the patient.

16. The apparatus of claim 1, said first electroactive polymer sleeve segment further comprising:
an annular ring, said annular ring comprising:
a perimeter about the first body part; and
a radius from a center of said annular ring to an inner side of said annular ring; and
a material comprising a shape change upon electrical stimulation,
wherein during use the electrical stimulation changes at least one of:
said perimeter by greater than ten percent;
said radius by greater than ten percent.

17. The apparatus of claim 16, said material comprising at least one of:
an ionic polymer-metal composite;
a dielectric polymer;
an electrostrictive polymer; and
a liquid crystalline polymer.

18. The apparatus of claim 16, further comprising:
a heart sensor, said heart sensor comprising at least one of a hemodynamic sensor and an electrodynamic sensor, said heart sensor configured to generate a signal related to a heart-beat of the patient,
said controller communicatively linked to said heart sensor,
said controller configured to send a signal to at least said first electroactive polymer sleeve dependent upon said signal.

19. The apparatus of claim 18, further comprising:
a first sleeve, said first sleeve comprising n electroactive polymer sleeve segments, where n is a positive integer of at least three, wherein two of said n electroactive polymer sleeve segments comprise said first electroactive polymer sleeve segment and said second electroactive polymer sleeve segment.

20. The apparatus of claim 19, further comprising:
a second sleeve comprising a set of m electroactive polymer sleeve segments, where m is a positive integer of at least three,
wherein said controller times constriction of said set of m electroactive sleeve elements and said set of n electroactive sleeve elements.

21. The apparatus of claim 19, said first sleeve comprising a sock configured for wearing by the patient.
